(19) United States
(12) Patent Application Publication
(10) Pub. No.: US 2014/0114384 A1
(43) Pub. Date: Apr. 24, 2014

(54) IMPLANTABLE FINE WIRE LEAD FOR ELECTROSTIMULATION AND SENSING

(71) Applicant: Cardia Access, Inc., Eden Prairie, MN (US)

(72) Inventors: Robert G. Walsh, Newport, OR (US); Jin Shimada, Grantsburg, WI (US)

(73) Assignee: Cardia Access, Inc., Eden Prairie, MN (US)

(21) Appl. No.: 14/056,819

(22) Filed: Oct. 17, 2013

Related U.S. Application Data
(62) Division of application No. 12/584,837, filed on Sep. 10, 2009, now abandoned.
Application No. 12/584,837 is a continuation-in-part of application No. 12/156,129, filed on May 28, 2008, now abandoned.
(60) Provisional application No. 61/191,722, filed on Sep. 10, 2008.

(51) Int. Cl. A61N 1/05

(52) U.S. Cl. CPC A61N 1/05
USPC 607/116

(57) ABSTRACT

A cardiac pacemaker or other implantable electrostimulation device has one or more durable fine wire leads to the heart or other electrostimulation site. The lead is formed of a core of silica or glass fiber or similar material, with a protective coating preferably including a metal buffer for conduction. The lead can be unipolar or bipolar (or even with three or more conductors), of small diameter and preferably with an anchoring configuration at the distal end of the lead. The anchor feature can take any of several nonlinear forms such that once implanted in a constrained configuration, the anchor can be released to the expanded, nonlinear configuration. The electrostimulation leads of the invention are extremely durable, can be bent through small radii and can exhibit long life without fatigue failure.

This application is a divisional of U.S. patent application Ser. No. 12/584,837 filed Sep. 10, 2009, which claims benefit from provisional application No. 61/191,722 filed Sep. 10, 2008; application Ser. No. 12/584,837 is further a continuation-in-part of U.S. patent application Ser. No. 12/156,129, filed May 28, 2008 (abandoned); each of these applications is incorporated herein by reference.

### BACKGROUND OF THE INVENTION

This invention concerns wiring for electrostimulation and sensing devices such as cardiac pacemakers, ICD and CRT devices, and neurostimulation devices, and in particular encompasses an improved implantable fine wire lead for such devices, a lead of very small diameter and capable of repeated cycles of bending without fatigue or failure. The invention also includes end anchors and electrodes on such leads.

The term therapeutic electrostimulation device (or similar) as used herein is intended to refer to all such implantable stimulation and/or sensing devices that employ wire leads.

Pacing has become a well-tested and effective means of maintaining heart function for patients with various heart conditions. Generally pacing is done from a control unit placed under but near the skin surface for access and communications with the physician controller when needed. Leads are routed from the controller to the heart probes to provide power for pacing and data from the probes to the controller. Probes are generally routed into the heart through the right, low pressure, side of the heart. No left, high pressure, heart access through the heart wall has been successful. For access to the left side of the heart, lead wires are generally routed from the right side of the heart through the coronary sinus and into veins draining the left side of the heart. This access path has several drawbacks; the placement of the probes is limited to areas covered by veins, leads occlude a significant fraction of the vein cross section and the number of probes is limited to 1 or 2.

Over 650,000 pacemakers are implanted in patients annually worldwide, including over 280,000 in the United States. Over 3.5 million people in the developed world have implanted pacemakers. Another approximately 900,000 have an ICD or CRT device. The pacemakers involve an average of about 1.4 implanted conductive leads, and the ICD and CRT devices use on average about 2.5 leads. These leads are necessarily implanted through tortuous pathways in the hostile environment of the human body. They are subjected to repeated flexing due to beating of the heart and the muscular movements associated with that beating, and also due to other movements in the upper body of the patient, movements that involve the pathway from the pacemaker to the heart. This can subject the implanted leads, at a series of points along their length, through tens of millions of iterations per year of flexing and unflexing, hundreds of millions over a desired lead lifetime. Previously available wire leads have not withstood these repeated flexings over long periods of time, and many have experienced failure due to the fatigue of repeated bending.

Neurostimulation refers to a therapy in which low voltage electrical stimulation is delivered to the spinal cord or targeted peripheral nerve in order to block neurosensation. Neurostimulation has application for numerous debilitating conditions, including treatment-resistant depression, epilepsy, gastroparesis, hearing loss, incontinence, chronic, untreatable pain, Parkinson's disease, essential tremor and dystonia. Other applications where neurostimulation holds promise include Alzheimer's disease, blindness, chronic migraines, morbid obesity, obsessive-compulsive disorder, paralysis, sleep apnea, stroke, and severe tinnitus.

Today's pacing leads manufactured by St. Jude, Medtronic, and Boston Scientific are typically referred to as multifilar, consisting of two or more wire coils that are wound in parallel together around a central axis in a spiral manner. This construction technique helps to reduce impedance in the conductor, and builds redundancy into the lead in case of breakage. The filar winding changes the overall stress vector in the conductor body from a bending stress in a straight wire to a torsion stress in a curved cylindrical wire perpendicular to lead axis. A straight wire can be put in overall tension, leading to fatigue failure, whereas a filar wound cannot. However, the bulk of the wire and the need to coil or twist the wires to reduce stress, limit the ability to produce smaller diameter leads.

Modern day pacemakers are capable of responding to changes in physical exertion level of patients. To accomplish this, artificial sensors are implanted which enable a feedback loop for adjusting pacemaker stimulation algorithms. As a result of these sensors, improved exertional tolerance can be achieved. Generally, sensors transmit signals through an electrical conductor which also serve as the pacemaker lead that enables cardiac electrostimulation. In fact, the pacemaker electrodes can serve the dual functions of stimulation and sensing.

It is an object of the invention described herein to overcome the problems of previously available implantable leads for electrostimulation and sensing, including pacemakers, ICD and CRT devices, and neurostimulation devices, with leads which are small in diameter and will exhibit very long-term durability. It is also an objective of this invention to detail novel structural and procedural features which are unique to the small diameter, high durability lead of this invention, and distinct from convention leads and lead systems.

### SUMMARY OF THE INVENTION

In the invention a flexible and durable fine wire lead for implanting in the body, connected to a pacemaker, ICD, CRT or other cardiac pulse generator, is formed from a drawn silica, glass, sapphire or crystalline quartz fiber core with a conductive metal buffer cladding. This cladding may be adhered directly on the core, or may be formed on top of one or more concentric or non-concentric layers of materials, termed intermediate layer(s), initially deposited on the core for purposes of aiding integrity and adhesion of the metal buffer cladding to the fine wire lead. The intermediate layer may consist of a suitable thin preferably hermetic (but possibly non-hermetic) coating of polymer or metal, or both. Depending on the degree of hermeticity of the intermediate layer or layers, the metal buffer cladding can be applied as a hermetic sealing material. In one process a polymer coating is first applied to the glass/silica fiber, then a first metal layer, followed by a second metal layer (or even further layers). The first layer can be one which bonds well to the polymer and aids bond integrity of the outer metal coating, but which is not an ideal conductor. The outer metal coating can be protective from the environment and of desired electrical conductivity.

Several different processes routinely associated with the fiber optics industry may be useful for creating both the intermediate layer, as well as the metal buffer cladding. These methods include coating glass fiber from a liquid solution of polymer or metal melt shortly after the fiber is drawn, various vapor deposition processes, electroplating, and electroless metallization. If metals are used as buffer or cladding, the metal may be a pure metal or an alloy.

One attractive approach is to fashion a cladding of graphene. This material provides a high strength cladding, and adheres well to a glass fiber core or to an intermediate layer adhered to the glass fiber core. Besides having exceptionally high strength, a graphene cladding is electrically conductive. An additional cladding consisting of metal and/or polymer can be used to cover the graphene. Such a coating can be designed to provide protection against host response. Suitable polymers might include silicon, polyurethane, polyimide or other biocompatible polymeric material.

There can additionally be a polymer coating over the metal buffer cladding, which may be biocompatible and resistant to environmental stress cracking or other mechanism of degradation associated with exposure and flexure within a biological system. The outer diameter of the fine wire lead preferably is less than about 750 microns, and may be 200 microns or even as small as 50 microns. Metals employed in the buffer can include aluminum, gold, platinum, titanium, tantalum, or others, as well as metal alloys of which MP35N, a nickel-cobalt based alloy, is one example. In a preferred embodiment the metal cladding is aluminum or gold, applied to the drawn silica, glass, sapphire or crystalline quartz fiber core immediately upon drawing and providing a protective hermetic seal over the fiber core.

If more than one conductor is needed, multiple unipole fibers can be used with one conductor per fiber. However, in another alternative the silica or other type fiber is used as a dielectric with a wire in the center of the fiber core as one conductor and the metallic buffer layer on the outside of the fiber core, both protecting the fiber and acting as the coaxial second conductor or ground return.

In a third embodiment, a further layer of silica, glass, etc. (as above) covers the metallic cladding, with a further electrically conductive buffer covering that dielectric layer. This embodiment may be with or without a center wire in the inner fiber. These silica, glass, etc. layers and buffer coatings can be continued for several more layers to produce a multiple conductor cable.

In a fourth embodiment the center of the fiber core is hollow to increase flexibility of a lead of a given diameter. In a fifth embodiment multiple conductors are embedded side-by-side in the silica, glass, etc. fiber core.

A further embodiment has the conductive lead composed of many smaller metal-buffered or metal wire-centered silica or glass fibers that together provide the electrical connection. This embodiment allows for high redundancy for each connection and very high flexibility.

The fiber coax is extremely strong and flexible. The current requirement for pacemaker leads does not dictate large central conductors, so that a few mils are sufficient (about 25 to 50 microns or so). The voltage used is very low, so insulator thickness requirements are minimal. The invention contemplates cables (meaning fiber-core leads with one or more conductors) of 100 to 200 micron diameter, and even unipolar cables as small as 50 microns in diameter or even smaller. These cables will have the flexibility to provide delivery to any portion of the heart.

Another advantage of using a coax fabricated from a silica/glass fiber insulator is that the connection between the cable and pacing probe (or a hub as discussed below) can be hermetic and therefore robust. Any portion of the fiber that is not protected from water or water vapor, such as in normal atmosphere, will rapidly degrade in strength due to the formation of surface cracks. This will allow that portion of the fiber to lose significant strength. Hermetically sealing the processed ends of the fiber cable will ensure that it remains rigid and protected, thus preserving the very high strength and fatigue resistance of the flexible portion of the fiber cable. Hermetic sealing is enabled by the use of an inorganic, high-temperature dielectric, glass or silica, which can be fused together with a similar dielectric, which is not the case with leads with organic materials. Hermeticity can be achieved whether the device is in the form of a coax or individual fibers cabled together, as long as an impervious surface seal is applied. This approach for applying a hermetic seal to the glass fiber lead can also be used for connection with industry standard connectors such as an IS-1, making the lead compatible with pacing systems from most manufactures.

The fiber coax is a combination of technologies that have been developed for different applications. Fiber cable is produced from a draw tower, a furnace that melts the silica or glass (or grown crystals for sapphire or quartz) and allows the fiber to be pulled, “drawn”, vertically from the bottom of the furnace. Fibers produced in this manner have strength of over 1 Mpsi. If the drawn fiber is allowed to sit in normal atmospheric conditions for more than a few minutes, that strength will rapidly be reduced to the order of 2-10 kpsi. This reduction is caused by water vapor attack on the outer silica or glass surface, causing minute cracking. Bending the fiber causes the outside of the bend to be put into tension and the cracks to propagate across the fiber causing failure. To ensure that the fiber remains at its maximum strength, a buffer is added to fibers as they are drawn. As the fiber is drawn and cools, a plastic coating, the buffer, is applied in a continuous manner protecting the fiber within a second of being produced.

The TOW missile was developed during the 1960s as an antitank missile for the U.S. Army. It included a fiber spooled from the rear of the missile as it flew. To further strengthen the fiber and protect it from damage, the typical plastic buffer was replaced with a metal buffer. The metal buffer used at that time was aluminum, but systems to coat fibers with gold and other metals have since been developed. The patents for the metal buffer technology covered a wide range of metals and alloys and were issued to Hughes in 1983 (U.S. Pat. No. 4,407,561 and U.S. Pat. No. 4,418,984).

The concept of using the fiber optical systems as a coax was developed for micro miniature x-ray sources by Xoft, Inc., Photoelectron Corporation and others. See U.S. Pat. Nos. 6,319,188 and 6,195,411. These fibers were used because they provided high flexibility, high voltage hold-off and direct connection to the x-ray source without a joint between the x-ray source and the HV power supply. The standard available optical fiber did not include a central electrical conductor. To include a wire in the center of the fiber, the wire must be drawn with the silica, glass, etc. fiber in the draw tower. For optical applications, to ensure that any optical energy launched into the fiber is not absorbed at the core wire interface, an additional lower index silica or glass cladding is provided between the core and the wire. All this is known prior practice.

Alternative methods of producing fiber coax include drawing a core fiber, coating that core with a metal buffer and drawing additional silica or glass over the assembly and cladding that final assembly with an additional metal buffer. Fibers can be pulled with a hole in the center as well, increasing flexibility; hole diameter can vary. In one embodiment one or more wires can be put inside the hole through a fiber. The fiber can be redrawn to engage the wire if desired.

Additional embodiments can also be used where the fiber, either solid core or hollow, can act as the strength member and dual electrical conductors can be placed outside the fiber system and separated by plastic or polymer insulators. Fatigue of metals and plastics after millions of small deflection stresses is one of the life-limiting aspects of conventional pacing leads. Silica, glass, etc. fibers protected with robust buffer systems will not exhibit fatigue. Fatigue in silica or glass is caused by propagation of cracks, which are present at low levels in typical silica or glass fibers as produced for standard communication purposes. Typically they exhibit only a few surface flaws per kilometer of fiber. Therefore silica or glass fiber coax cables make ideal pacing leads: small diameter, low mass, highly flexible, robust and with very long service life.

One method according to the invention for testing fibers for leads is to stretch a long segment until it breaks; the weakest point in the fiber will break first. If the fiber meets some minimal standard for tensile strength, then the entire fiber meets that strength minimum and flaws will not exist up to some level. If the fiber does break, the remaining pieces can be similarly tested. As this is repeated the limits at which the fiber will break will continue to climb, allowing selection of extremely flaw-free sections of fiber. This will further enhance the ability of the fiber to resist failure due to repeated stress cycling; the fiber does not fatigue from exposure to tensile loading. This is a type of fiber “proofing”, but proofing as previously known was for lot testing rather than for selections of sections of highest strength from a fiber. Pursuant to the invention fibers for use in the fine wire leads are proofed to at least about 90% of the intrinsic tensile strength value of the material, or more broadly, at least about 75%.

The invention described herein also contemplates a pacing system that avoids problems encountered in prior systems and provides more versatility than previously available. In this system a control hub is implanted in the pericardia region of the heart. The hub is connected to each pacing site by a silica, glass, etc. cable conductive lead of one of the types described above. The hub in turn is connected to the pacemaker by a single lead using the same technology. The hub system provides for shorter, localized connections involving a plurality of conductive leads, often five or six or more, while only one lead need be implanted between the hub and the pacemaker, normally implanted higher in the chest and just under the skin. The hub or pacing can could also serve as a depot for electronics that would process sensing information received from electrodes attached distally to the hub, in order to select which leads receive stimulation. This has implications for situations in which a first lead shows signs of dysfunction; the electronics can be switched by the physician to stimulate a different lead. Alternatively, if the desired location of stimulation changes chronically due to physiological changes in the heart, electronics can sense this and provide the physician with information needed to enable selection of a different lead for stimulation.

The glass/silica fine wire lead of the invention is compatible with drug/steroid elution for controlling fibrosis adjacent to the lead electrode, which is a known technique used with conventional pacing leads for controlling impedance and thus battery life. For example a bioerodable polymer can be positioned on the distal end of a lead at the electrode, the polymer containing the eluting drug.

The invention thus encompasses an improved, highly durable fine wire electrostimulation/sensing lead, achieved by incorporation of several unique structural and procedural features, which enable successful placement of the small diameter durable glass fiber lead of the present invention into a desired location within the coronary venous system. In particular, the invention includes a procedure by which the glass is metallized to produce the glass fiber lead. In addition, the means by which the lead is delivered to the desired venous location, requiring a suitable delivery system adapted to the unique structural features of the lead is described.

The fine wire leads of the invention can employ anchoring systems for stabilizing the fiber lead against unwanted migration within the coronary vein. Such anchoring systems can consist of expandable/retractable stents attached to the lead, or helical, wavy, angled, corkscrew, J-hook or expandable loop-type extensions attached to the lead, that take on the desired anchoring shape after delivery of the lead from within a delivery catheter.

Delivery devices can be used for installation of the fine wire leads of the invention. A steerable catheter for example, can be used and then removed when the leads are properly deployed in the proper anatomical positions.

It is among the objects of the invention to improve the durability, lifetime flexibility and versatility of wire leads for pacemakers, ICDs, CRTs and other cardiac pulse generators, as well as electrostimulation or sensing leads for other therapeutic purposes within the body. These and other objects, advantages and features of the invention will be apparent from the following description of preferred embodiments, considered along with the accompanying drawings.

### DESCRIPTION OF THE DRAWINGS

FIG. 1 is a schematic drawing showing one embodiment of an anchoring means for an implantable fine wire lead for a cardiac pulse generator such as a pacemaker.

FIG. 2 is a similar view showing another embodiment of an anchoring means for a fine wire lead.

FIG. 3 is a view showing a further embodiment of an anchoring means for a fine wire lead.

FIG. 4 is a view showing another embodiment of an anchoring means for a fine wire lead incorporating a terminal loop structure containing electrodes.

### DESCRIPTION OF PREFERRED EMBODIMENTS

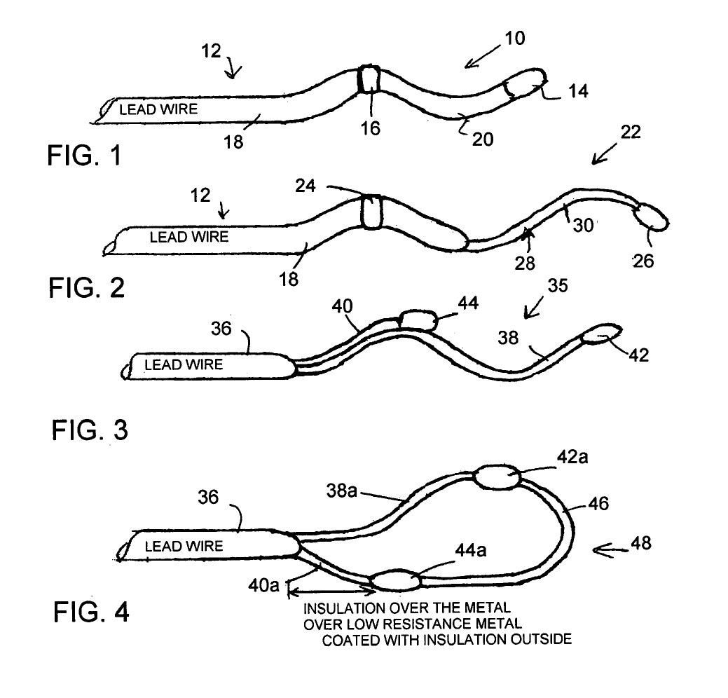

FIG. 1 is a simple representation of one preferred embodiment of the tip or distal portion 10 of an implantable fine wire lead 12 pursuant to the invention, and which may be formed similar to the leads shown in patent application Ser. No. 12/156,129. The lead wire is a drawn silica or glass fiber core (which is intended to include sapphire or crystalline quartz) with a conductive metal buffer cladding over the fiber core. The metal cladding is in turn coated with an insulative material such as a polymer, ceramic, glass, or carbon coating. Two or more of these constructions can then be combined to produce the lead wire 12 as illustrated in FIG. 1, with two conductors. Surface electrodes 14 and 16 are incorporated, each in electrical contact with a separate metal clad glass fiber within the lead wire 12. Insulative material is applied as a top coat 18 to the lead wire in such a way that a non-linear pattern is created. This may be a generally sinusoidal shape as depicted, although other shapes including helical or screw patterns, angled fish hook, coil, zig-zag, Y-shaped, or other non-linear patterns are envisioned as well. The shape can be formed in the lead tip, then the insulative coating applied so that the shape is maintained. The electrodes 14 and 16 are positioned so that contact is facilitated with the inner surface of a coronary vein in which the lead wire is positioned. Contact points for the electrodes 14 and 16 may be on the same aspect of the vein inner surface as depicted in FIG. 1, or may be on opposite aspects of the vein inner surface. This will depend on the shape of the lead wire tip and the relative positioning of electrodes 14 and 16 on the lead wire.

The distal tip of the glass fiber lead is considered to be bipolar in FIG. 1. This essentially means that the electrical conductors for the two electrodes are envisioned to run through the complete length of the lead. The two conductors are insulated from each other, with one conductor connecting with the electrode 14 and one conductor connecting with the electrode 16. The electrical conductor for electrode 16 need not extend further distally than its point of contact with the electrode 16. Other components of the composite lead 12, including one of the glass fibers and a biocompatible outer polymer coating 20, extend completely to the distal tip of the lead 12 in such a way that the overall diameter of the lead can remain relatively constant throughout the distal tip region of the lead 12.

Various means are envisioned for fashioning an anchor feature to the tip of the silica/glass fiber lead. The feature can be added as a separate tip component to the fiber lead. This involves a suitable connector that supplies structural stability as well as electrical conductivity to each electrode of the tip component. Such a connector is envisioned to be a cylindrical coupling with a central orifice in which the distal end of the glass/silica fiber is inserted in one end and the tip component is inserted into the other end. The distal tip component may be envisioned to have electrodes built into it, or may have electrodes added to it after sealing the distal tip to the glass fiber. The components are then sealed through one of various processes depending on materials.

Sealing methods include polymerization of a space-fill polymer, such as polyurethane or polyimide, or application of heat, which would serve to anneal components with each other. As an alternative to a cylindrical coupling, a female coupling can be built into the proximal end of the distal tip component for mating with the glass fiber lead. A connector similar to that shown in FIG. 8 of patent application Ser. No. 12/156,129 can be used, with a complementary female connector. Sealing may be by similar means as described above. Another way of providing the shape feature to the distal end of the glass fiber to provide the anchoring device is to insert the distal end of the glass fiber lead into a heating block which has been routed out to provide the desired three-dimensional shape to the distal tip of the glass fiber lead. Heat is then supplied to the heating block to anneal the distal tip to a non-linear shape. This step may be accomplished either before or after insertion and sealing of electrode elements into the distal tip region of the glass fiber lead. In addition, the distal tip portion of the glass fiber lead may be placed in a shaping mandrel in which a non-linear track is fashioned to provide the desired non-linear shape to the distal tip portion of the glass fiber lead. One of several polymers is then molded around the glass fiber tip within the shaping mandrel to lock the distal tip of the lead in a non-linear configuration. This step may be accomplished either before or after incorporating electrodes into the distal tip of the glass fiber lead. In either case, steps in the molding process are taken to insure that electrodes have direct exposure to cardiac venous tissue and are not covered by polymer. The polymer coating may or may not provide a hermetic seal.

As noted above, the non-linear anchor feature can be implanted in a restrained condition (as in a sheath), then released to the expanded anchor shape when correctly positioned.

Another approach to providing a non-linear anchoring feature at the distal end of the glass fiber lead is to incorporate a separate non-linear element into the distal tip construction. This non-linear element, likely fashioned out of a metal such as nitinol, stainless steel, or other suitable metal or metal alloy, is positioned to extend parallel with the distal tip of the glass fiber lead. It is insulated by a dedicated polymer sleeve, or by incorporation into a polymer coating encompassing the non-linear element along with the glass fiber distal tip.

FIG. 2 shows a modification of the configuration described in FIG. 1, with a different anchor feature 22 at the end of a lead wire 12. In FIG. 2 the bipolar construction of electrical conductors within the glass/silica lead terminates at or near a first electrode 24, where one of the two electrical conductors makes contact with that electrode. Insulating polymer 18 may extend beyond the electrode 24, but terminates somewhere between the electrodes 24 and an end electrode 26. A second single electrical conductor 28, preferably covered and insulated, then extends beyond the termination of the first electrical conductor, making contact with the tip electrode 26. This extension also has an outer biocompatible polymer layer 30, in which polyurethane or silicon are two examples used routinely for such applications. The diameter of the most distal portion of the glass/silica fiber lead, containing a unipolar electrical conductor, may be smaller than the diameter of the main body of the glass fiber lead 12 which contains the bipolar electrical conductors. The anchor feature of FIG. 2 is generally sinusoidal in shape but has an additional curve as compared to FIG. 1, so that the two electrodes are at opposite sides or aspects of a vein.

FIG. 3 is a similar view, showing a bipolar lead construction with an anchor feature 35. Distal ends of each of the bipolar leads, with associated terminal electrodes, extend beyond a common outer biocompatible polymer housing 36. One or both of the extensions 38 and 40 may be non-linear, according to methods specified previously. The extension 28 and 40 are of different lengths, so that the respective electrodes 42 and 44 are positioned within a coronary vein with a dimensional offset in the axial direction. As in the other constructions described, the tip electrode or most distal electrode 42 is typically the electrostimulation electrode while the more proximal electrode 44 is the sensing or return electrode.

FIG. 4 represents a modification of the construction shown in FIG. 3, in which an additional extension 46 of non-conducting material is inserted to connect the electrodes 42 and 44a, forming a loop anchor 48. This non-conductive insertion 46 may consist of polymer-coated nitinol, or may consist of one or more polymers, intended to provide non-linear, loop shaping to the electrode elements, while providing acceptable biocompatibility. As above, the non-linearity facilitates anchoring of the distal electrode elements against the vein wall.

In each of the anchor configurations described above, the anchor feature can be in three dimensions rather than in only two.

One method for forming the electrodes in the described embodiments is by direct coating of metal onto the lead wire, which can be by dipping the lead into molten metal. This step can be incorporated as a final coating step in which a non-conductive insulative polymer is applied to all surface area of the buffered lead, except where the electrode is located. Alternatively the entire lead can be coated with a polymer (e.g., immediately after drawing the fiber), hermetically covering the fiber, and a portion can be burned away or otherwise removed later when the electrode metal is applied.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

What is claimed is:

1. A method comprising:
providing a fine wire electrostimulation lead of glass or silica construction of sufficiently small size to be capable of implanting in a coronary blood vessel, with the electrostimulation lead including a protective layer being formed as a coating around the glass or silica construction and with the conductor being formed as a coating around the polymer layer;
configuring a distal end on the electrostimulation lead as an anchor to retain the distal end in place as implanted in a vessel by engaging against the walls of a vessel and configuring the distal end as a non-linear, curving shape which when unconstrained occupies a greater diameter than that of the vessel, the distal end being integral in the electrostimulation lead and with the glass or silica construction of the electrostimulation lead being continuous into the distal end, the distal end including an electrode connected to the conductor, the distal end including a shape memory metal or polymer extending through at least a portion of the distal end so that the distal end tends to assume the non-linear configuration when unconstrained, and the distal end being capable of being constrained to a small diameter when retained in an implanting catheter such that the fine wire electrostimulation lead is implantable into the vessel via a catheter; and
providing or positioning the electrostimulation lead with the distal end such that when the distal end is implanted in the vessel the electrode contacts a wall of the vessel.

2. The method of claim 1, wherein the electrostimulation lead includes a cladding adhered to the glass or silica construction via or along with the protective layer.

3. The method of claim 2, wherein the electrostimulation lead includes a conductive metal layer around the cladding, and further includes passing current through the conductive metal layer and to the electrode to provide electrostimulation at the wall of the vessel, wherein the outer diameter of the electrostimulation lead is greater than 50 microns and not greater than 200 microns.

4. An apparatus, comprising:
a fine wire electrostimulation lead of glass or silica construction of sufficiently small size to be capable of implanting in a coronary blood vessel, the fine wire including a conductor, a distal end on the fine wire lead configured and arranged as an anchor to retain the distal end in place as implanted in a vessel by engaging against the walls of a vessel, the distal end being formed into a non-linear, curving configuration which when unconstrained occupies a greater diameter than that of the vessel, the distal end including an electrode connected to the conductor, positioned such that when the distal end is implanted in the vessel the electrode contacts a wall of the vessel, the distal end including a shape memory metal or polymer extending through at least a portion of the distal end so that the distal end tends to assume the non-linear configuration when unconstrained, and the distal end being capable of being constrained to a small diameter when retained in an implanting catheter such that the fine wire electrostimulation lead is implantable into the vessel via a catheter.

5. The apparatus of claim 4, wherein nitinol is included in the distal end as said shape memory metal.

6. The apparatus of claim 4, wherein the non-linear, curving configuration comprises one of the following: wavy, loop, corkscrew, J-hook, coil, helix, forked.

7. The apparatus of claim 4, wherein the distal end in the non-linear, curving configuration includes two electrodes isolated from one another within the lead and in positions in the non-linear configuration to promote contact with a vessel wall.

8. The apparatus of claim 4, wherein the non-linear, curving configuration is non-planar.

9. The apparatus of claim 4, wherein the distal end is integral in the fine wire electrostimulation lead, with the glass or silica construction of the lead continuous into the distal end.

10. The apparatus of claim 4, wherein the distal end with the anchoring feature is a separate tip component connected to the glass or silica electrostimulation lead.

11. The apparatus of claim 4, further comprising a cardiac electrostimulation device connected to the lead.

12. The apparatus of claim 4, wherein the electrostimulation lead includes a solid core configured and arranged to provide flexibility of the fine wire electrostimulation lead.

13. The apparatus of claim 4, wherein the electrostimulation lead includes a first portion and a second portion connected by a connector that is configured and arranged to provide a continuous communication path for electrostimulation.

14. The apparatus of claim 4, wherein the electrostimulation lead includes a first portion including a first electrode at one end of the first portion, a second portion including a second electrode at one end of the second portion, and a third portion connecting the first electrode and the second electrode, the third portion forming the distal end of the fine wire electrostimulation lead.

15. The apparatus of claim 14, wherein the third portion connecting the first electrode and the second electrode forms a loop.

16. An apparatus, comprising:
a fine wire electrostimulation lead of glass or silica construction of sufficiently small size to be capable of implanting in a coronary blood vessel, the fine wire including a conductor, a distal end on the fine wire lead configured and arranged with the distal end being integral in the fine wire electrostimulation lead and with the glass or silica construction of the lead being continuous into the distal end, and configured and arranged as an anchor to retain the distal end in place as implanted in a vessel by engaging against the walls of a vessel, the distal end being formed into a non-linear, curving configuration which when unconstrained occupies a greater diameter than that of the vessel, the distal end including an electrode connected to the conductor, positioned such that when the distal end is implanted in the vessel the electrode contacts a wall of the vessel, and the distal end including a shape memory metal or polymer extending through at least a portion of the distal end so that the distal end tends to assume the non-linear configuration when unconstrained, and the distal end being capable of being constrained to a small diameter when retained in an implanting catheter such that the fine wire electrostimulation lead is implantable into the vessel via a catheter.
